(19) United States
(12) Patent Application Publication
(10) Pub. No.: US 2025/0044300 A1
(43) Pub. Date: Feb. 6, 2025

(54) METHOD, DEVICE AND COMPUTER SOFTWARE FOR PREPARING A LIQUID PROTEIN SAMPLE FOR PROTEOME ANALYSIS

(71) Applicant: Thermo Fisher Scientific (Bremen) GmbH, Bremen (DE)

(72) Inventors: Roman Zubarev, Stockholm (SE); Zhaowei Meng, Stockholm (SE)

(73) Assignee: Thermo Fisher Scientific (Bremen) GmbH, Bremen (DE)

(21) Appl. No.: 18/783,842

(22) Filed: Jul. 25, 2024

(30) Foreign Application Priority Data
Jul. 31, 2023 (GB) 2311745.0

(51) Int. Cl. G01N 33/68

(52) U.S. Cl. CPC G01N 33/6848

(57) ABSTRACT

A method of preparing a liquid protein sample for proteome analysis is provided. The method comprises pumping a liquid protein sample into a heated tubing, wherein the liquid protein sample comprises a plurality of portions; incubating the liquid protein sample in the heated tubing, wherein an incubation condition for a first portion of the sample is different to an incubation condition for a second portion of the sample; and pumping the liquid protein sample out of the heated tubing.

### CROSS REFERENCE TO RELATED APPLICATION

This application claims priority from application GB 2311745.0, filed Jul. 31, 2023. The entire disclosure of application GB 2311745.0 is incorporated herein by reference.

### TECHNICAL FIELD

The present invention relates to the field of proteomics. Specifically, the invention provides an improved method for preparing a liquid protein sample for proteome analysis.

### BACKGROUND

Proteins are linear chains of amino acids, and these chains often acquire their biological function upon folding into compact three-dimensional structures. As proteins are building blocks of cellular structure and indispensable elements of cellular mechanics, most processes that occur in a cell or organism induce changes in three-dimensional structures of protein involved in these processes. When cells or isolated proteins interact with other molecules, including other proteins, nucleic acids, metabolites and drug molecules, protein structures undergo changes. Monitoring these changes is important to understand how cells live, metabolise food, proliferate, communicate with each other, mutate, differentiate and die.

In proteomics, it is often desired to understand how an active molecule or molecules (e.g. a drug) affects each different protein within a proteome.

There are a number of prior art methods that perform analysis of changes in protein structure for isolated, highly purified proteins, such as thermal shift assay, circular dichroism, hydrogen-deuterium mass spectrometry, nuclear magnetic resonance and X-ray crystallography. There are also some recent prior art methods to monitor structural changes in individual proteins in complex mixtures and at the level of the whole proteome. One such method is Stability of Proteins from Rates of OXidation (SPROX), (West et al., “Quantitative proteomics approach for identifying protein-drug interactions in complex mixtures using protein stability measurements,” PNAS, 107 (20): 9078-9082 (2010)). This reference and all others cited are incorporated herein by reference. In this approach, hydrogen peroxide is added to the proteome at different concentrations to oxidize the methionine residues in target proteins. The methionine oxidation levels depend upon the three-dimensional protein structure and are measured in each sample by mass spectrometry. SPROX has been successfully used to identify the targets of small molecules, such as cyclosporine A. One disadvantage of SPROX is the relatively low frequency of methionine residues in the peptides. Another disadvantage is that hydrogen peroxide induces undesirable side reactions in the protein.

Another method of probing changes in protein three-dimensional structure in non-purified samples is based on limited proteolysis (Piazza et al., “A machine learning-based chemoproteomic approach to identify drug targets and binding sites in complex proteomes,” Nat. Commun. 11 (4200) (2020)). In the method described, the proteome is partially digested by a protease, and the digestion rates for different cleavage sites are monitored by proteomics. Changes in protein three-dimensional structure due to different processes induce variations in the cleavage rates for the sites involved in these processes. This method is technically difficult and hard to automate, because the protein digestion rates depend upon a number of experimental parameters that are difficult to control.

Thermal Proteome Profiling, TPP, (Savitski et al., “Tracking cancer drugs in living cells by thermal profiling of the proteome,” Science 346 (6205): 55 & 1255784-1-1255784-10 (Oct. 3, 2014)) is a method for probing protein structure at the proteome level. FIG. 1 illustrates a Thermal Proteome Profiling (TPP) method, as described in Savitski et al.

The assessed parameter in this method is the protein thermal stability which is probed by changing the temperature of the proteome solution from normal (typically, 37 degrees Celsius) to elevated, such as 67 degrees Celsius or higher. At elevated temperatures, many proteins change their conformation from natively folded to denatured, or melted, conformation. Thermal profiling methods are based on measuring the protein melting temperature, which is postulated to increase when the protein binds a ligand. The disadvantage of the thermal profiling methods is that they require splitting the protein sample into as many fractions (and reaction vessels) as there are temperature points, typically ten or more points. This approach, therefore, requires a significant volume of sample for testing and may lead to sample losses (e.g., through manual pipetting). Also, since each fraction is incubated at a different temperature, it is technically challenging to ensure high reproducibility of such analysis.

Proteome Integral Solubility Alteration, PISA, (Gaetani et al., “Proteome Integral Solubility Alteration: A High-Throughput Proteomics Assay for Target Deconvolution,” Journal of Proteome Research, 18:4027-4037 (Nov. 1, 2019)) provides a method for measuring protein expression and thermal stability changes in cells. During sample preparation, the cell suspension is distributed into multiple different PCR tubes and each is heated to a temperature in a given temperature range. The samples are then combined together and the protein concentrations are measured. Unlike in TPP, in PISA the measured solubility alterations are not necessarily interpreted as thermal stability changes, because, as noted in this document, the relation between solubility and stability is complex.

FIGS. 2A-2G illustrate the PISA method described in this document. During sample preparation, the sample is split into equal portions for treatment at different temperatures. The samples are subsequently pooled into a final mixture for analysis. PISA may therefore be used to reduce MS analysis time but still requires multiple vessels for sample treatment.

A method related to thermal profiling uses organic solvent instead of temperature for denaturing proteins (Zhang et al., “Solvent-Induced Protein Precipitation for Drug Target Discovery on the Proteomic Scale,” Anal. Chem., 92:1363-1371 (Jan. 7, 2020)). Yet another method uses an increased salt concentration (Beusch et al., “Ion-Based Proteome-Integrated Solubility Alteration Assays for Systemwide Profiling of Protein-Molecule Interactions,” Anal. Chem., 94:7066-7074 (May 2022)). The advantage of both these methods is that they are isothermal and may be performed at room temperature. The disadvantage is that these methods are difficult to automate, while their manual implementation is labour-intensive and can result in variability of the final results. These methods also require significant amount of sample, because treatment of each fraction proceeds in a different vessel.

Existing methods for probing a proteome all require significant manual steps during sample preparation and are therefore prone to errors and inconsistencies. Therefore, an improved method of sample preparation for proteome analysis is required.

### SUMMARY

A method of preparing a liquid protein sample for proteome analysis is provided. The method comprises:
pumping a liquid protein sample into a heated tubing, wherein the liquid protein sample comprises a plurality of portions;
incubating the liquid protein sample in the heated tubing, wherein an incubation condition for a first portion of the sample is different to an incubation condition for a second portion of the sample;
pumping the liquid protein sample out of the heated tubing.

Pumping a liquid protein sample into a heated tubing may be referred to as “filling” the heated tubing. Pumping the liquid protein sample out of the heated tubing may be referred to as “eluting” the liquid protein sample.

In contrast to the TPP and PISA methods described in the prior art, the invention incubates one quantity of sample in a heated tube, so that different portions of the sample experience different incubation conditions. This obviates the need for separating out the sample for incubation at different temperatures. As a result, the incubation conditions are applied more consistently between samples, compared to manual separation and incubation of samples. The results obtained are therefore more reliable.

Moreover, prior art methods that require manual separation and incubation of samples often require a larger quantity of sample overall. Therefore, the method enables more efficient use of the sample.

One important difference between the method and the prior art is that the portions of sample do not require physical separation into different PCR tubes, in order to achieve the required incubation conditions. Unlike the TTP or PISA methods, One-Pot Time-Induced (OPTI)-PISA provides different incubation conditions for different portions of sample by pumping the sample through a uniformly heated tubing at a carefully controlled speed or by subjecting the tubing to a temperature gradient. In either case, the sample prepared by the method is not physically separated and is not incubated separately. This is nowhere disclosed in any of the prior art documents, which all describe that the sample is separated and incubated at different temperatures (and, in the case of PISA, pooled together for analysis).

The disclosed invention circumvents the problems of the prior art methods by performing incubation in a single vessel, allowing for sample volume reduction and easy automation of the whole incubation process.

The invented assay can be useful in several different areas of biology and medicine. One application is the determination of the protein to which drug molecule or another ligand binds. Commonly, such proteins are called target proteins. Particularly, during the development of chemical compounds into drugs, it is important to know which protein the drug interacts with. When protein target is known, it is important to verify that the compound interacts with the drug target at given conditions and in a given environment. The target identification and monitoring of target protein-ligand interactions can therefore be used in initial screening for interacting ligands from large chemical libraries, as well as during optimization of an initial ligand into a candidate drug.

In other drug development applications, such a phenotypic screening, where the desired action of a drug candidate on living cells or cellular lysates is discovered, an important task is to determine which of the thousands of proteins in the cell or lysate interacts with the drug and thus which protein is the target protein for a given drug.

Furthermore, it is important to identify the interaction of a drug with other proteins (so called “off-target interactions”) where such interactions may result in undesirable side effects of treatments. Such side effects may also be desirable, in which case the drug can be repurposed to treat a different condition than was originally planned.

Because of the complexity of processes affecting a drug in a living organism, it is important to verify that the drug that binds the target protein in a cellular assay also interacts with it in a living organism. Therefore, it is desirable to be able to detect ligand-protein interactions in complex non-purified samples, e.g. bodily liquids, cell lysates, cells or biopsies from experimental animals or patients. Furthermore, it is an advantage to have the analysis method isothermal, performed in a single vessel without sample splitting, and automated as much as possible. The present disclosure provides a method for solving this difficulty by performing PISA in a single capillary.

In a first embodiment, the tube is heated to a constant temperature. In this embodiment, the method and apparatus are further simplified by performing incubation at a constant temperature (a single temperature can be provided using a heated plate, without complex equipment for establishing a temperature gradient). In this embodiment, the different portions of sample are incubated at the same temperature but with different incubation times. The pumping speed of the liquid sample is controlled to regulate the incubation time of different portions of the sample.

The incubation condition may comprise a period of time during which the respective portion of the sample is incubated in the heated tubing.

The heated tubing may be heated to a predetermined temperature.

The entirety of the heated tubing may be held at a single predetermined temperature.

Each of the plurality of portions may be incubated in the heated tubing for a different period of time.

A filling speed, being a pumping speed for pumping the liquid protein sample into the heated tube, may be significantly different from an elution speed, being a pumping speed for pumping the liquid protein sample out of the heated tube, so that each of the plurality of portions is incubated in the heated tubing for a different period of time.

The “speed” may be expressed as a flow rate in some examples. In other examples, this may be the speed at which the fluid travels along the tubing. The flow rate may be calculated as the speed multiplied by the cross-sectional area of the heated tubing.

The filling speed and/or elution speed may be varied during filling and/or elution, so that each of the plurality of portions is incubated in the heated tubing for a different period of time.

Incubating the sample in the tubing may comprise pumping the sample through the heated tubing at a controlled speed, so that an incubation period is defined as the time taken for the respective portion of the liquid protein sample to pass from an inlet of the heated tubing to an outlet of the heated tubing.

The controlled speed need not be a constant speed. Indeed, so that each different portion of the liquid protein sample experiences a different incubation period, the speed may not be constant. For example, the heated tubing may be filled with sample at a relatively high speed and then the sample may be eluted from the heated tubing at a relatively low speed, or vice-versa.

Each of the plurality of portions may be incubated in the heated tubing for a different period of time on average, such that an average incubation time for a first portion of sample is different to an average incubation time for a second portion of sample.

In other words, the variation in incubation time for sample eluting from the tube (sample spatially separated along the length of the tube) may be continuous. Nevertheless, a portion of the sample having a defined volume may have an associated average incubation time. The average incubation time may be calculated by the integral of the integration time over the volume and divided by the volume of the sample.

In a second embodiment, a temperature gradient is applied to the tube, so that each portion is incubated at a different temperature. A second embodiment of the invention involves maintaining a temperature gradient at the heated tubing and holding the sample within the tube for a time period, so that all of the sample is incubated for the same amount of time but different portions are incubated at different temperatures.

The incubation condition may comprise a temperature at which the respective portion of the sample is incubated in the heated tubing.

The heated tubing may be heated to a temperature gradient or a plurality of different temperatures.

Each different temperature may be applied to a respective portion of the heated tubing, each portion of the heated tubing may correspond to a portion of the liquid protein sample held therein.

Incubating the sample in the heated tubing may comprise holding the sample stationary in the tubing for a predetermined incubation period between the steps of pumping the sample into the tubing and pumping the sample out of the tubing.

The first and second portions may be incubated in the heated tubing at a different temperature on average.

The sample may be pumped into and out of the heated tube quickly. Nevertheless, the sample may need to travel through each part of the heated tube to get from the inlet to the outlet. Therefore, the incubation temperature for a given portion of the sample will not be constant, as the portion travels through the temperature gradient/different temperatures applied by the heated tube. However, once the heated tubing has been filled, the sample may be held stationary in the heated tube for a period of time. Therefore, the average incubation temperature for each portion will be approximately equal to the temperature applied to that portion when the sample is stationary.

The incubation condition may be different for each of the plurality of portions.

In other words, in addition to the first and second portions being subjected to different incubation conditions, the incubation conditions for each and every portion may be different from the incubation conditions for every other portion.

The sample may comprise at least three portions, preferably at least five portions and more preferably around ten portions.

A “heated” tubing may be interpreted as a tubing that is actively heated above room temperature.

The method may further comprise pumping the liquid protein sample into an unheated tubing in fluid communication with the heated tubing.

Pumping the liquid protein sample into the heated tubing and/or pumping the liquid protein sample out of the heated tubing may comprise pumping washing solvent into the unheated tubing.

Since the heated tubing and the unheated tubing are in fluid communication, pumping washing solvent into the unheated tubing may cause the liquid protein sample in the unheated tubing to be pushed out of the unheated tubing and into the heated tubing. Pumping further washing solvent into the unheated tubing may cause the washing solvent in the unheated tubing to be pushed out of the unheated tubing an into the heated tubing, thus pushing the liquid protein solution out of the heated tubing.

The method may further comprise creating an air bubble in the unheated tubing between the liquid protein sample and the washing solvent.

The air bubble may keep the washing solvent separate from the liquid protein sample to isolate the liquid protein sample and prevent contamination.

The method may further comprise cooling the liquid protein sample after it has been pumped out of the heated tubing. Cooling the liquid protein sample may help to stabilise it so that further heat-induced changes to the proteins in the sample are limited.

The method may further comprise analysing the liquid protein sample to determine a relative abundance of one or more proteins in the sample.

The liquid protein sample may be analysed using an analytical instrument such as a mass spectrometer.

Preferably, the portions of the treated sample are analysed together (in a PISA method). Alternatively, the different portions of the sample could be separated as different fractions and analysed separately (in a TPP method).

Pumping the liquid protein sample out of the heated tubing may comprise pumping the liquid protein sample out of the heated tubing in a plurality of fractions.

Each of the plurality of fractions may correspond to a portion of the liquid protein sample, so that an incubation condition for a first fraction of the sample is different to an incubation condition for a second fraction of the sample.

The method may further comprise analysing each fraction of the liquid protein sample separately to determine a relative abundance of one or more proteins in each fraction.

The liquid protein sample may comprise a protein mixture and/or living cells.

The method may further comprise adding one or more test compounds to the liquid protein sample prior to incubating the liquid protein sample in the heated tubing.

A device configured to perform a method described above is also provided. The device comprises:
a heated tubing,
a pump configured to pump a liquid protein sample into a heated tubing and pump the liquid protein sample out of the heated tubing.

The device may further comprise one or more of:
a fraction collector configured to collect the liquid protein sample pumped out of the heated tubing, and
an analytical instrument configured to analyse the liquid protein sample pumped out of the heated tubing.

Computer software is also provided. The computer software comprises instructions that, when executed on a processor, cause the processor to perform a method as described above.

Further examples useful for understanding the invention are set out in the following numbered clauses:

Clause 1. A method for detecting target proteins for a compound or compounds added to a non-purified complex protein mixture, comprising:
(a) Preparing liquid sample consisting of protein mixture or living cells with or without added test compound or compounds;
(b) Filling with said sample a tubing that is kept fully or partially at a fixed temperature;
(c) Eluting said sample from said tubing after a period of time ranging from zero to several minutes.

Clause 2. The method according to clause 1, wherein the elution speed of the sample is made significantly different from the filling speed, so that different volumes of the sample are exposed to said temperature for different time intervals.

Clause 3. The method according to clause 1, wherein the filling speed or elution speed or both speeds are changing during filling or elution, so that different volumes of the sample are exposed to said temperature for different time intervals.

Clause 4. The device utilizing method according to clause 1, comprising at least following modules: one autosampler, one pump, one tubing kept at a constant temperature, and one fraction collector, with autosampler containing protein samples and pump enabling filling said tubing with protein samples and their elution from tubing to fraction collector.

Clause 5. The device according to clause 4, in which all mentioned modules are programmable in the software or user interface, and all operation steps are programmed in the software or user interface prior to running samples.

Additionally, an instrument for automatically executing the methods of this invention is described.

### BRIEF DESCRIPTION OF THE DRAWINGS

FIG. 1 illustrates a Thermal Proteome Profiling (TPP) method.

FIGS. 2A-2G illustrate a Proteome Integral Solubility Alteration (PISA) method.

FIG. 3A illustrates example apparatus and flow of liquid protein sample though the example apparatus.

FIG. 3B illustrates a graph of flow rate of liquid protein sample against time for the apparatus illustrated in FIG. 3A.

FIG. 4 illustrates a graph of incubation time for a portion of sample against the position of the sample in the tubing for the apparatus of FIG. 3A and the flow rate of FIG. 3B.

FIG. 5A illustrates another example apparatus.

FIG. 5B illustrates a graph of flow rate of liquid protein sample against time for the apparatus illustrated in FIG. 5A.

FIG. 6 illustrates results from a specific example method.

### DETAILED DESCRIPTION

One method of understanding how an active molecule or molecules (e.g. a drug) affects each different protein within a proteome is “Thermal Proteome Profiling” (TPP), which relies on changes in protein thermal stability to assess how the molecule(s) affects different proteins within a proteome. As described in Savitski et al., this involves:
dividing a sample into many fractions,
heating each fraction to a different temperature (in some examples, 10 different fractions for each of 10 different temperatures are used), and
determining a “melting curve” for each protein in the sample.

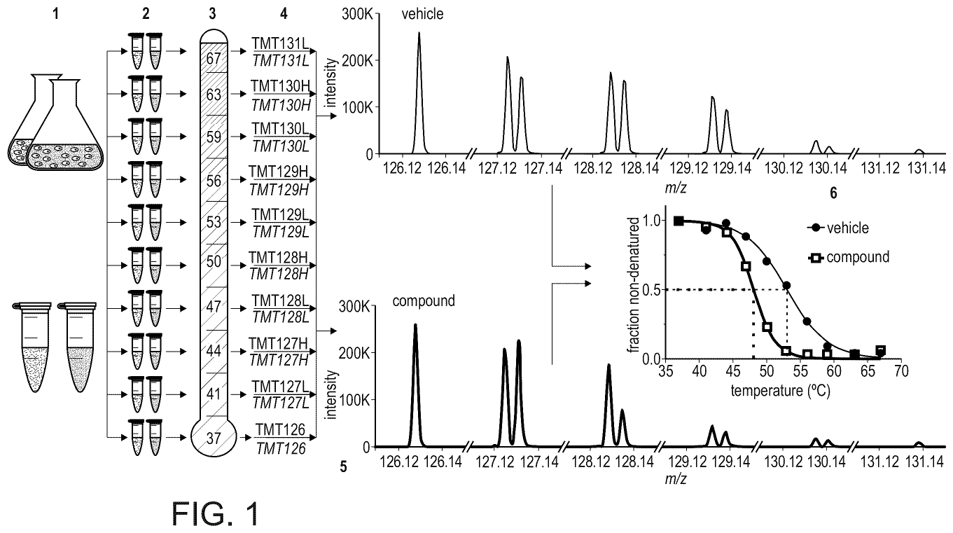

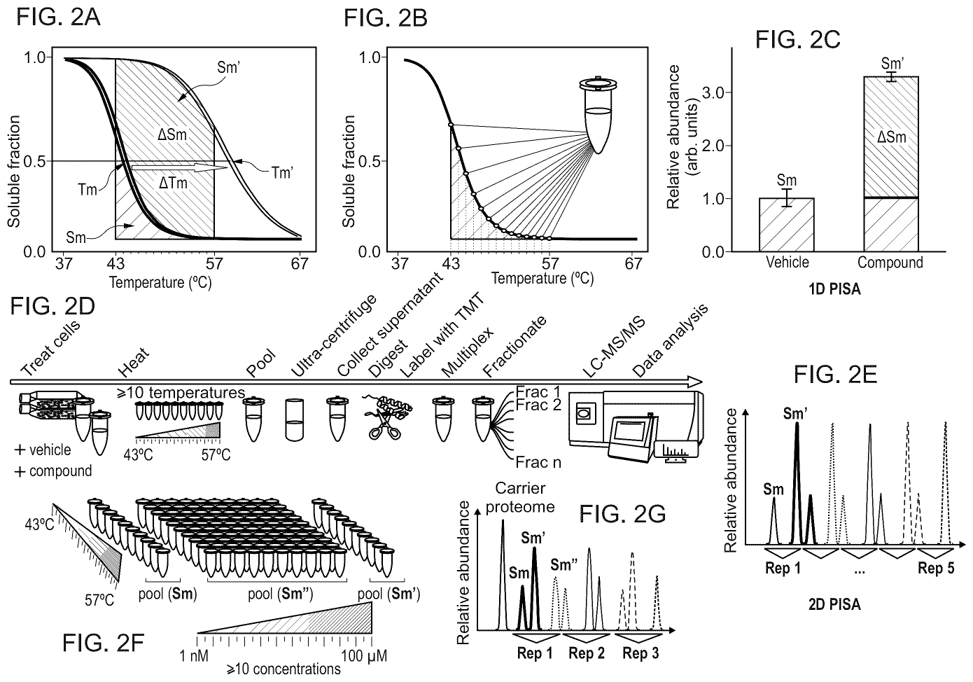

A melting curve is a plot of the protein's abundance versus temperature (see FIG. 1).

Typically, the melting curves of “target” proteins that bind to the molecule(s) are shifted to higher temperatures compared to those of proteins that do not bind to the molecule(s). For example, this may occur because the thermal stability of the protein increases when bound to the molecule(s).

A related method is “Proteome Integral Solubility Alteration” (PISA) assay, examples of which are described in Gaetani et al. and Sabatier et al., which provides an alternative to TPP in which the sample may be analysed together. This may therefore improve the throughput of the proteome profiling method.

Some example methods enable analysis of changes in protein three-dimensional structure by providing a differential solubility assay in which two or more proteome states are compared in terms of the differences in solubility of individual proteins in these states. As most proteins exist in the cell in both soluble and insoluble state (Vecchi et al., “Proteome-wide observation of the phenomenon of life on the edge of solubility,” PNAS, 117 (2): 1015-1020 (Jan. 14, 2020)), almost all known proteomics protocols include the step of separating the soluble and insoluble parts of the proteome. Most proteomics approaches are based on measuring the abundances of the soluble proteins as a measure of protein concentration in the sample. Soluble protein abundance may be used as a measure of protein solubility in a given proteome state.

In order to ensure that not all molecules of a given protein are fully soluble at any proteome state and thus to increase the dynamic range of the solubility measurements, part of the sample is incubated at a different temperature compared to ambient. Many proteins become less soluble at a higher temperature, but some proteins lose their solubility and a temperature near freezing point. Moreover, related proteins may have different solubility properties if they come from different organisms. Therefore, the optimal temperature of incubation will depend upon the specifics of the proteins of interest.

The invention describes a method and apparatus for commercial automation of the TPP or PISA method. The original TPP and PISA methods involve dividing a sample into many (e.g. ˜10) fractions and incubating each fraction at a different temperature. This is relatively complex, time-consuming manual task, and uses a relatively large amount of sample (e.g. ˜10×˜10 μL).

In prior art TPP and PISA methods, incubation occurs at different temperatures but for the same time duration. In contrast, the invention obtains the same (or similar) information using only one quantity of sample (“one-pot” operation), in a manner which is significantly less complex and that is suitable for automation.

In some examples, instead of relying on multiple different temperatures, the method relies on different incubation times. The time of incubation is different for different portions of the liquid protein sample. In such examples, the incubation temperature may be one fixed temperature.

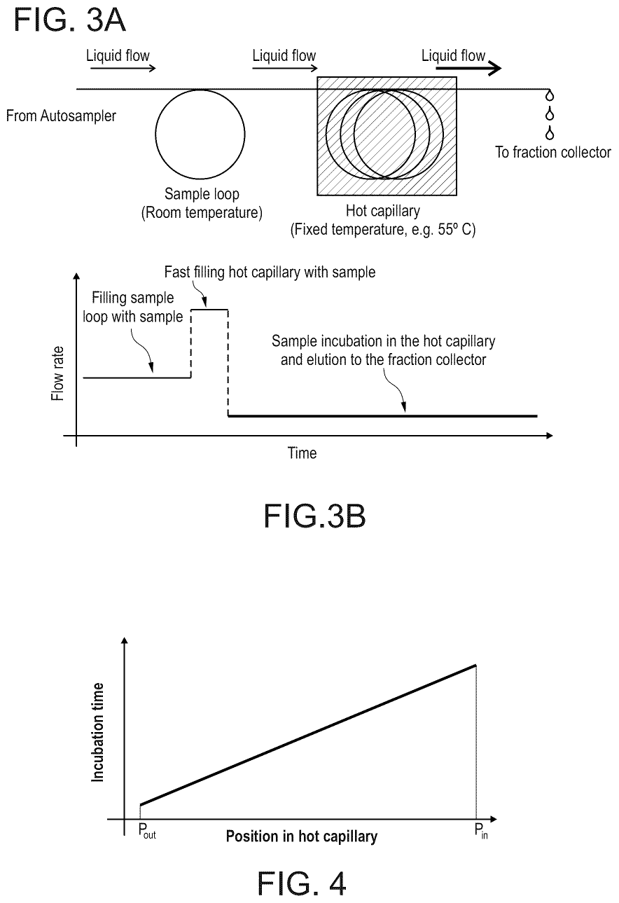

In order to achieve this, the volume in which incubation occurs is elongated spatially, such as in a tubing or capillary, and different parts of the sample are exposed to a fixed temperature for different periods of time. FIG. 3A illustrates example apparatus and flow of liquid protein sample through the heated tubing to implement some example methods. FIG. 3B illustrates a graph of flow rate of liquid protein sample against time for some example methods. FIG. 4 illustrates a graph of incubation time for a portion of sample against the position of the sample in the tubing.

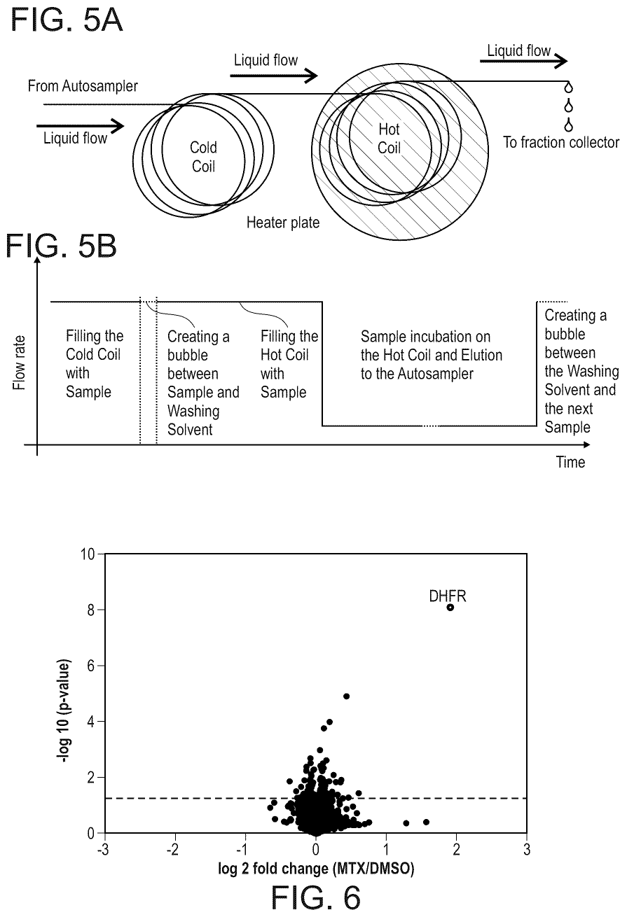

Another example apparatus is illustrated in FIG. 5A. FIG. 5B illustrates a graph of flow rate of liquid protein sample against time for other example methods.

In a first example, a sample is loaded into a long capillary tube which is heated at a fixed temperature, e.g. by being attached to a heated plate (“hot coil”). The speeds at which the sample is loaded into and removed (eluted) from the tube are separately controllable by one or more pumps. The sample is loaded into the heated tube during a relatively short time-period (e.g. during a ˜10 s period). Then, the sample is eluted from the heated tube during a relatively long time-period (e.g. over a ˜10 min. period). Those portions of the sample that are eluted from the heated tube relatively early-on in the experiment will have been incubated for a short amount of time, while those portions of the sample that are eluted from the heated tube relatively late-on in the experiment will have been incubated for a large amount of time. Then, by analysing the different portions of the sample which have experienced different incubation times, “melting curves” can be obtained for each protein in the sample.

It has been shown that the method can be automated, and works well using small amounts of sample (e.g. ˜0.9 μg).

In a second example, a long capillary tube could be attached to a heated plate having two (or more) heaters held at different temperatures, so that a temperature gradient is maintained across the plate/capillary. All of the sample could be retained in the capillary for the same period of time. Different parts of the sample inside the capillary would experience different temperatures due to the temperature gradient.

Although the abundance of each protein is preferably measured using mass spectrometry, other techniques could in principle also or instead be used, such as western blotting, mass photometry, etc.

One known problem with the method is cross-contamination of samples (due to sample sticking to the walls of the tube). This can be addressed by washing and/or passivating the tube before each measurement.

Instead of a single heated plate, two heated plates could be used, e.g. with the tube sandwiched between them. Alternatively, a wire heated could be wrapped around the tube.

In one specific example, a device comprises:
A) An Autosampler containing Input wells with liquid samples of protein mixtures or living cells and a Needle capable of collecting defined volumes of said liquid sample. The Autosampler may have a cooling system keeping the sample stable before processing in the rest of the device by refrigerating them.
B) A Pump capable of transferring said volumes from said Needle to Tubing kept at a constant temperature by, for example, attaching it to a heated Plate made of material with high thermal capacity, such as metal or ceramic. It is advantageous to make Tubing also from material with high thermal capacity, such as fused silica.
C) An electronic temperature Controlling System capable of maintaining fixed temperature of said Plate elevated compared to the ambient temperature.
D) A Fraction Collector capable of receiving liquid sample volumes eluting through said Tubing and collecting them into Output wells.
E) A Software Program operating said Needle, Pump, Controlling system and Fraction collector.

One specific example method comprises the following steps:
a) Preparation step: preparing liquid samples containing cell lysates or intact cells with or without the molecule of interest, such as drug, and depositing prepared sample into a given Input well of Autosampler.
b) Treatment step: using Pump, transferring said liquid sample from a given Input well into Tubing and then eluting liquid sample from Tubing to Fraction collector such that different volumes of sample were exposed to temperature at which Tubing is kept for different time periods. This can be achieved either by different constant rate of filling in Tubing compared to constant rate of elution from Tubing, or by using varying rate of filling Tubing, or by using varying rate of eluting sample from Tubing, or by combination thereof.
c) Collection step: using Fraction collector, deposit eluting sample into an Output well corresponding to the Input well from which a given sample originates.
d) Lysing step (only for samples containing intact cells): lysing intact cells and obtaining cell lysate.
e) Centrifugation step: Centrifugating each sample lysate and separating soluble proteins from insoluble proteins.
f) Analysis step: analyzing by quantitative proteomics soluble proteins obtained in the Centrifugation step.
g) Data processing step: Comparing protein relative abundances and determining proteins that changed their abundance, and thus their solubility, in presence of drug.

Some example methods may be used during analysis of individual proteins and protein mixtures, including proteins in unfractionated cell lysates and intact cells. Further example methods may be used in assessing the changes in the protein solubility due to thermal unfolding. One application area is the determination in a non-purified complex sample of proteins that interact with a test compound of interest added to the mixture.

The readout of the differential solubility assay in the simplest case of two proteome states is the difference or ratio of the abundances between these two states for every protein. Such measurements performed in a number of replicates provide statistical significance of the difference or ratio via an appropriate statistical test, such as Student's t-test.

FIG. 6 illustrates results from a specific example. Cell lysate was treated with methotrexate (MTX) and for control with vehicle (DMSO). 5 μL of samples in 5 replicates were placed in an autosampler. Automatic syringe loaded 3 μL of samples one by one into an OPTI-PISA capillary at high speed (<1 min). After a short time samples were slowly eluted from the OPTI-PISA capillary into a fraction collector (≥10 min). Collected samples were digested, TMT labeled and analyzed by LC-MS/MS. MTX target DHFR was identified as an outlier in a volcano plot with excellent p-value (<10⁻⁸). Only 900 ng of lysate was used per sample. The results indicate a better p-value than in an equivalent manual PISA experiment. The improvement may be attributed at least in part to automation of the sample incubation.

The described OPTI-PISA methods therefore provide a number of advantages. Some methods enable full automation of thermal treatment for PISA experiment in lysates. In some embodiments, the sample consumption is low compared to prior art methods, below 1 μg per sample in some examples. Some example methods therefore provide high reproducibility of results, due to reliable automation. The results may therefore provide a better (lower) p-value than in manual PISA experiments on the same samples. Some example methods may result in treatment times of approximately 10-15 minutes per sample. Therefore, the described methods provide a high throughput compared to prior art methods. The described methods may also be compatible with automated proteome analysis equipment and software.

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that, for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise.

Unless otherwise defined, all other technical and scientific terms used herein have the meaning commonly understood by one of ordinary skill in the art to which this invention belongs. In case of conflict, the present specification, including definitions, will control. It will be appreciated that there is an implied “about” prior to the quantitative terms mentioned in the present description, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. As used herein, “a” or “an” also may refer to “at least one” or “one or more.” Also, the use of “or” is inclusive, such that the phrase “A or B” is true when “A” is true, “B” is true, or both “A” and “B” are true.

1. A method of preparing a liquid protein sample for proteome analysis, the method comprising:
pumping the liquid protein sample into a heated tubing, wherein the liquid protein sample comprises a plurality of portions;
incubating the liquid protein sample in the heated tubing, wherein an incubation condition for a first portion of the liquid protein sample is different from an incubation condition for a second portion of the liquid protein sample; and
pumping the liquid protein sample out of the heated tubing.

2. The method of claim 1, wherein the incubation condition comprises a period of time during which the respective portion of the liquid protein sample is incubated in the heated tubing.

3. The method of claim 1, wherein the heated tubing is heated to a predetermined temperature.

4. The method of claim 1, wherein each of the plurality of portions is incubated in the heated tubing for a different period of time.

5. The method of claim 1, wherein a filling speed, being a pumping speed for pumping the liquid protein sample into the heated tubing, is significantly different from an elution speed, being a pumping speed for pumping the liquid protein sample out of the heated tubing.

6. The method of claim 5, wherein one or more of the filling speed and the elution speed are varied during filling and/or elution, so that each of the plurality of portions is incubated in the heated tubing for a different period of time.

7. The method of claim 1, wherein incubating the liquid protein sample in the heated tubing comprises pumping the liquid protein sample through the heated tubing at a controlled speed, so that an incubation period is defined as a time taken for the respective portion of the liquid protein sample to pass from an inlet of the heated tubing to an outlet of the heated tubing.

8. The method of claim 1, wherein each of the plurality of portions is incubated in the heated tubing for a different period of time on average, such that an average incubation time for a first portion of sample is different to an average incubation time for a second portion of sample.

9. The method of claim 1, wherein the incubation condition comprises a temperature at which the respective portion of the liquid protein sample is incubated in the heated tubing.

10. The method of claim 9, wherein the heated tubing is heated to a temperature gradient or a plurality of different temperatures.

11. The method of claim 9, wherein incubating the liquid protein sample in the heated tubing comprises holding the liquid protein sample stationary in the heated tubing for a predetermined incubation period between the steps of pumping the liquid protein sample into the heated tubing and pumping the liquid protein sample out of the heated tubing.

12. The method of any of claim 9, wherein the first and second portions are incubated in the heated tubing at a different temperature on average.

13. The method of claim 1, wherein the incubation condition is different for each of the plurality of portions.

14. The method of claim 1, further comprising pumping the liquid protein sample into an unheated tubing in fluid communication with the heated tubing.

15. The method of claim 14, wherein pumping the liquid protein sample into the heated tubing and/or pumping the liquid protein sample out of the heated tubing comprises pumping washing solvent into the unheated tubing.

16. The method of claim 15, further comprising creating an air bubble in the unheated tubing between the liquid protein sample and the washing solvent.

17. The method of claim 1, further comprising cooling the liquid protein sample after it has been pumped out of the heated tubing.

18. The method of claim 1, further comprising analysing the liquid protein sample to determine a relative abundance of one or more proteins in the liquid protein sample.

19. The method of claim 1, wherein pumping the liquid protein sample out of the heated tubing comprises pumping the liquid protein sample out of the heated tubing in a plurality of fractions.

20. The method of claim 19, further comprising analysing each fraction of the liquid protein sample separately to determine a relative abundance of one or more proteins in each fraction.

21. The method of claim 1, wherein the liquid protein sample comprises a protein mixture and/or living cells.

22. The method of claim 1, further comprising adding one or more test compounds to the liquid protein sample prior to incubating the liquid protein sample in the heated tubing.

23. A device configured to perform the method according to claim 1, the device comprising:
a heated tubing,
a pump configured to pump the liquid protein sample into a heated tubing and pump the liquid protein sample out of the heated tubing.

24. The device of claim 23, further comprising one or more of:
a fraction collector configured to collect the liquid protein sample pumped out of the heated tubing, and
an analytical instrument configured to analyse the liquid protein sample pumped out of the heated tubing.

25. A computer-readable medium having stored thereon processor-executable instructions for performing the method of claim 1.
